(19) United States
(12) Patent Application Publication
(10) Pub. No.: US 2018/0078191 A1
(43) Pub. Date: Mar. 22, 2018

(54) Medical Toilet for Collecting and Analyzing Multiple Metrics

(71) Applicants: David R. Hall, Provo, UT (US); Dan Allen, Springville, UT (US); Min Kang, Provo, UT (US); Ben Swenson, Lehi, UT (US); Terrece Pearman, Draper, UT (US)

(72) Inventors: David R. Hall, Provo, UT (US); Dan Allen, Springville, UT (US); Min Kang, Provo, UT (US); Ben Swenson, Lehi, UT (US); Terrece Pearman, Draper, UT (US)

(21) Appl. No.: 15/270,674

(22) Filed: Sep. 20, 2016

(51) Int. Cl. A61B 5/20; A61B 5/0205; A61B 5/024; A61B 5/021; A61B 5/053; A61B 5/00; G06F 19/00

(52) U.S. Cl. CPC A61B 5/207; A61B 5/0205; A61B 5/024; A61B 5/021; A61B 5/0531; G06F 19/3406; A61B 5/4875; A61B 5/746; A61B 5/7275; A61B 5/0022; G06F 19/3431; A61B 5/208

(57) ABSTRACT

The present invention is directed to a toilet which is a medical device and which includes multiple sensors and mechanisms for analyzing health metrics that may include biomarkers. The toilet is in communication with a computer processer that is programmed to evaluate the validity of collected health metrics. One set of metrics may inform the validity of another set of metrics. Each data point within the multiple health metrics is assigned a weight value that is an indicator of its validity. Data points may be excluded from calculations if deemed to be invalid. Data points may also be flagged as requiring additional information about the user to determine its validity or interpretation. Methods of using the toilet to assess the health status of a user is also disclosed.

### BACKGROUND

### Field of the Invention

This invention relates to systems for determining health conditions.

### Background of the Invention

Convenient ways to measure health data are often less accurate than more invasive alternatives. However, accuracy can be a challenge for certain body types or individuals with certain physical conditions that need to be monitored. This may be because the health parameter is often being inferred from the results of an analytical technique rather than directly measured. Additionally, health care providers sometimes simultaneously use multiple methods to infer an individual's health status, each associated with a different degree of accuracy and relevance to the individual, in an attempt to create a complex health assessment or to select a single diagnosis out of a lengthy differential diagnosis. The shortcoming of each measurement may be taken into account when interpreting the data generated by the measurement. Furthermore, many clinical measurements are merely snap shots of an individual's health status on a particular day. However, daily measurements of multiple physiological processes to create a complete health assessment, using only data that is likely to be valid, is often be prohibitive. A way to determine which measurement is the most accurate and meaningful to use in making assessments and decisions about an individual's health is needed. Furthermore, a method to conduct measurements of multiple physiological parameters daily, or multiple times each day, with an assessment of each measurement's validity is also needed.

### BRIEF SUMMARY OF THE INVENTION

We disclose a novel health metering device and methods of use thereof to identify which health measurements are accurate and relevant to assess a user's health. More specifically, we disclose a device and methods to collect multiple health data measurements, hereinafter, “metrics,” including a mechanism to combine, filter, and/or cull the collected metrics. The device includes a computer that is programmed to provide output calculations and reports that a healthcare provider may use to assess the user's health status. While a plurality of metrics may be collected at any one time, some metrics may be weighted relative to others, a process which indicates the relative validity of each metric. In addition, metrics may be calculated differently based on the body type or health status of the user, each of which may be identified by one or more of the metrics. Thus, the disclosed device may be used to individually tailor reported health data based on the body type or other physical parameters of the individual user. The most accurate and meaningful data is therefore reported or flagged as useful data. In contrast, less meaningful data may be omitted from the report or identified as not relevant to the particular user's health status. In the instant disclosure, the metrics include those that are conducted by a toilet that is a medical device. The toilet referenced herein measures multiple metrics then transmit the metrics to a computer that is programmed to process the metrics based on their validity and relevance to the individual user's health status.

### BRIEF DESCRIPTION OF THE DRAWINGS

FIG. 1 is a perspective view of one embodiment of a toilet communicating at least two measurements to a network, cell phone, and computer in accordance with an embodiment of the invention.

FIG. 2 is a front view showing a user sitting on the toilet of FIG. 1 while metrics are collected and communicated to a network in accordance with an embodiment of an invention.

FIG. 3 is a flow chart illustrating an embodiment of a decision making process for analyzing heart rate data from a user in accordance with an embodiment of the invention.

FIG. 4 is an isometric view of a toilet showing various sensors and health measurement devices in accordance with an embodiment of the invention.

FIG. 5 is an isometric view of a toilet showing various sensors and health measurement devices in accordance with an embodiment of the invention.

FIG. 6 is a flow chart showing how bioimpedance data from multiple sets of sensors may be evaluated and selected or rejected in accordance with an embodiment of the invention.

FIG. 7A is a flow chart showing how bioimpedance data is valued based on user movement.

FIG. 7B is a flow chart showing how bioimpedance data is valued based on completion of urination.

FIG. 8 is a flow chart illustrating an example in which an EKG measurement may indicate atrial fibrillation.

FIG. 9 is a flow chart that illustrates an example in accordance with an embodiment of the invention in which daily urine glucose measurements are taken.

### DETAILED DESCRIPTION OF THE INVENTION

### Definitions

Toilet, as used herein, means a device that collects biological waste products of a mammal including urine, feces, and vomit.

Metric, as used herein, means a system, method, or standard of measurement.

Data means information, numerical or otherwise, that is collected using one of a variety of health measurement methods.

Health status, as used herein, means the current physiological state of a mammal. In general, this term refers to the overall health of the mammal. However, individual parameters relating to a specific body part or biological system may be measured to identify disease states or physiological parameters that are outside of those known in the art to be within normal range. Such individual physiological parameters may be used to define the health status of the mammal with regard to a specific physiological system.

User, as used herein, means any mammal, human or animal, for which the toilet disclosed herein is being used to measure physiological functions.

While this invention is susceptible of embodiment in many different forms, there are shown in the drawings, which will herein be described in detail, several specific embodiments with the understanding that the present disclosure is to be considered as an exemplification of the principals of the invention and is not intended to limit the invention to the illustrated embodiments.

Disclosed herein is a health metering device and methods for use thereof which provides assessment of the validity and relevance of the metrics it collects. Multiple metrics which either directly indicate or infer a user's health status are collected. Some of these metrics may provide an indication of the validity of others. Each metric is assigned a weight value based on the values of other metrics taken at the same or different time points. Metrics that have been assigned a weight value below a threshold value may be flagged as invalid or excluded from multi-variable calculations that provide an assessment of the user's health status.

In the embodiments disclosed herein, the health metrics are collected as a user interacts with a toilet. These health metrics may be collected while the user is simply sitting on the toilet, standing in front of the toilet on a scale associated with a toilet, and or while the user is depositing bodily waste into the toilet. The toilet comprises multiple sub-devices which measure different physiological parameters then transfer the metrics to a computer for processing according to programming as disclosed herein.

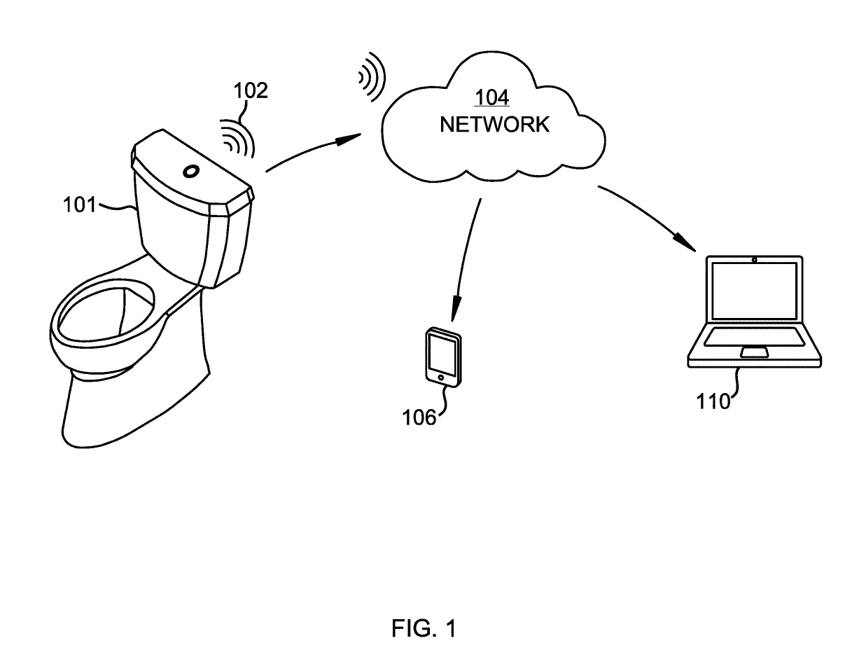

Referring now to the figures, FIG. 1 illustrates one embodiment of a toilet 101 that is communicating metrics that it collected from a user via a wireless signal 102 to a data collection system which may comprise a network database 104 and a data communication port. This data is accessible via a cell phone 106 or computer 110. Note that toilet 101 is capable of measuring two or more metrics that may be relevant to assess the user's health status. Alternatively, a first metric may be relevant to the user's health status and the second metric may be useful to assess the validity of the first metric. When the two or more metrics have been collected, the metrics are transferred to network database 104 via wireless signal 102. While the embodiment in FIG. 1 transfers data via wireless signal 102, other embodiments may include one or more data communication ports which may be connected to an Ethernet, a local computer, or a flash drive to collect, store, and transfer collected metrics. In each of these embodiments, the metrics are subjected to calculations and/or assessment by a processor, which may be a computer. The metrics may be transferred to a server that a user's health care provider may access. This may be done via, for example, Cloud technology when the user performs a predetermined action such as pressing a button. Alternatively, the metrics may be transferred to the health care provider's server immediately following collection.

The two or more metrics that toilet 101 may measure include, but are not limited to, body temperature, body weight, body composition (i.e. percent body fat, intracellular and/or extracellular water), heart rate, pedal pulse rate, blood pressure, blood oxygen saturation, electrocardiogram (EKG or ECG) measurement, urine constituents and parameters including urine color, glucose, urea, creatinine, specific gravity, urine protein, electrolytes, urine pH, osmolality, human chorionic gonadotropin (for detecting pregnancy), hemoglobin, white blood cells, red blood cells, ketone bodies, bilirubin, urobilinogen, free catecholamines, free cortisol, phenylalanine, and urine volume. The metrics may also include fecal analysis including fecal weight and volume, calprotectin, lactoferrin, and hemoglobin. Additionally, one or more of the flow, volume, and weight sensors may determine periods of excretion activity to measure, for instance, urination or defecation exertions, or selectively record metrics that were collected during periods of low exertion. These metrics are useful because some metrics are best performed after complete voiding of the bowel and bladder for maximum accuracy.

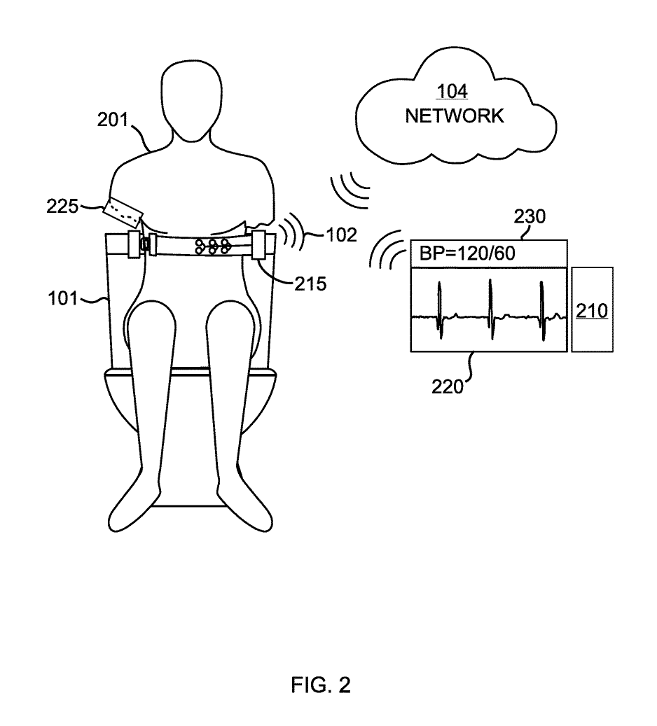

FIG. 2 is a front view illustrating an individual 201 during a health measurement session. The individual may be urinating in toilet 101 which may perform various analyses on the urine. A health measurement device 215 is shown gathering heart rate data 220 and blood pressure data 230 is being measured by device 225. The data is being communicated via the wireless signal 102 to the network database 104.

The heart rate data 220 and blood pressure data 230 collected for the individual shown in FIG. 2 may be outside of normal ranges which may infer a compromised health status. However, the analysis of the same individual's urine may indicate dehydration. By processing the data such that the dehydration status of the individual is taken into consideration, the abnormal heart rate data 220 and blood pressure data 230 may be interpreted in context and assigned a lower weight with regard to the individual's cardiovascular health. A set of measurements taken at another time, when the individual is properly hydrated, may then be used to give a more accurate health status assessment. Thus, the validity of first metric (heart rate and/or blood pressure) is assessed by the second metric (urine analysis).

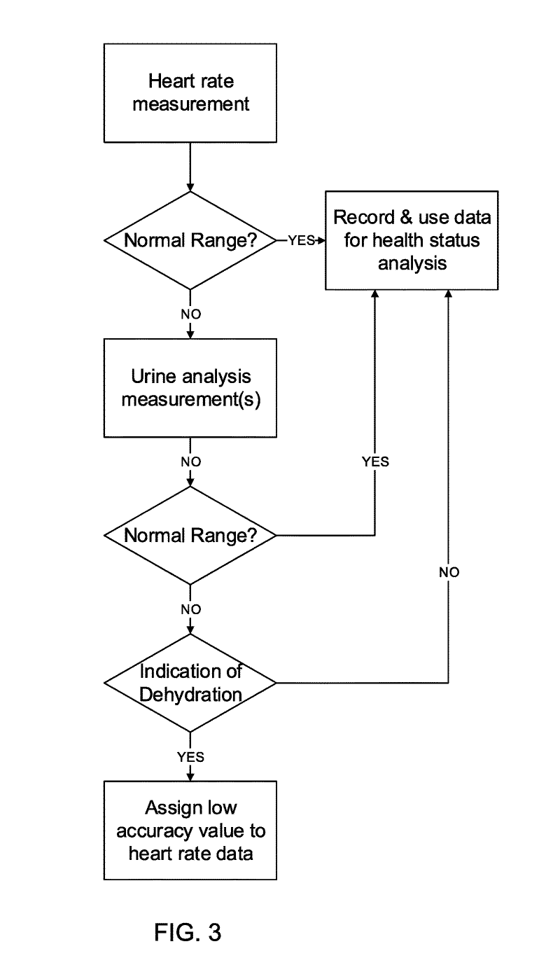

FIG. 3 is a flow chart illustrating the decision process that may be used to interpret and assign relevance to metrics collected using toilet 101 or other embodiments thereof. In the illustrated situation, sensors on toilet 101 detect an abnormal heart rate. At approximately the same time, or shortly after, toilet 101 analyzes the user's urine. If certain abnormal values are collected from the analysis of the user's urine, the computer processor determines that the values suggest that the user is dehydrated. If the user is dehydrated, this could be the reason for an abnormal heart rate. The heart rate metric is then assigned a lower validity value (which could be numerical). Later analysis of multiple health data points may exclude this abnormal heart rate measurement as it may not be a valid indication of the user's health status and simply a measurement taken after the user was dehydrated due to an event such as a period of heavy exercise. A metric of the user's heart rate taken when an accompanying urine analysis suggests that the user is sufficiently hydrated will be given a higher weight value and thus rated as a more valid metric.

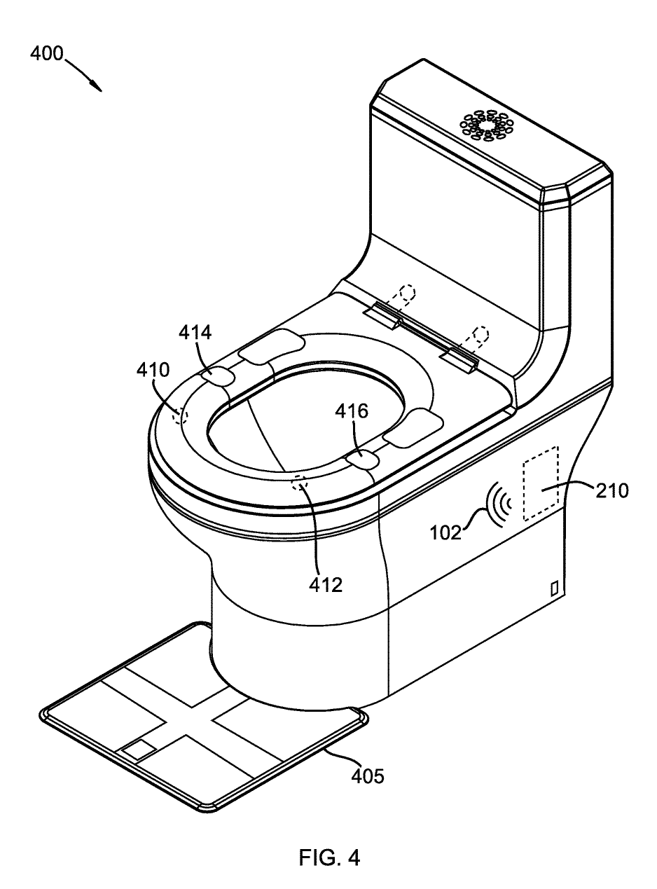

FIG. 4 is an isometric view of a toilet 400 with multiple health measurement sub-devices shown. All of the sub-devices communicate the collected metrics to processor 210. In this embodiment, wireless signal 102 communicates the metrics to the network, as in the description of FIG. 1. A scale 405 is shown along with pressure sensors 410 and 412 in the seat all of which collect weight measurements. Bioimpedance sensors 414 and 416 are in contact with the individual's skin to identify body composition including total body water. The processor may use these metrics perform calculations to assess the individual's percent body fat. This information may be stored and used to inform, and thereby weight, the relevance and interpretation of other metrics. For example, if the individual's height is provided, the body weight measurement may be used to calculate a body mass index (BMI) which is weight expressed in kilograms divided by height squared in meters (BMI=Weight/(Height)²). An extremely healthy and fit athlete with a low percent body fat may have a high BMI and erroneously be interpreted to be unhealthy. But, according to the invention disclosed herein, the data indicating that the individual has a low percent body fat will assign a lowered weight (relevance) to the BMI calculation.

The embodiment of toilet 400 shown in FIG. 4 may include a flow meter or liquid level meter. Note that the seat includes pressure sensors 410 and 412 and there is a scale 405 with pressure sensors in front of toilet 400. The user's weight may be determined by the sum of measurements provided from pressure sensors 410, and 412 when the user is seated on the toilet, and from scale 405 when the user's feet are placed on scale 405.

The computer processor may be programmed to report the measured body weight after urination or defecation is complete. The flow or level meter data indicates when urination or defecation is complete or nearly complete by detecting that the level measured is approximately constant at that time or by detecting that the flow measured is small.

The invention disclosed herein may be used to compile an accurate trend of physiological changes over time. The health trend reporting system, for example, that disclosed in U.S. patent application Ser. No. 15/242,929 filed on Aug. 22, 2016 may combine multiple weight measurements to report the weight trend to the user or to the user's health care provider. The weighting of each measurement may be different depending on a second measurement parameter. The computer program that calculates and reports the body weight trend may preferentially weight measurements that were taken when the user's total body water level is within a defined range. As discussed above, total body water can be estimated by either bioimpedance. It may also be estimated by properties of the urine such as specific gravity, color, or urea or creatinine concentration.

In summary, the estimate of hydration level of a user by either urinalysis or bioimpedance may allow appropriate calculations to be selected to estimate changes in body fat or body weight over time by giving more weight to measurements taken when the user is properly hydrated. One of skill in the art will readily understand that the example of using urinalysis to assess bioimpedance metrics is merely an example and that the instant invention may be applied to other health metric sets in an analogous way.

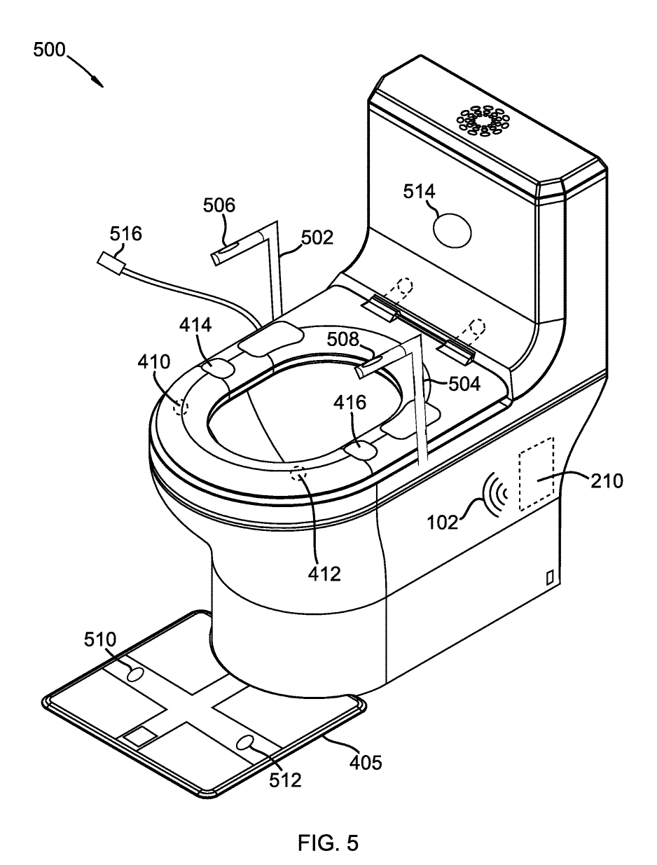

FIG. 5 illustrates toilet 500 an embodiment that is a variation of toilet 400 of FIG. 4. Traditionally, impedance measurements have used electrodes to measure the impedance from hand to foot, foot to foot, hand to hand, or thigh to thigh among other methods. The optimal method may depend on factors such as gender and obesity. Thus, metrics that measure parameters such as body weight and input that a health care provider may directly enter into the computer processor, such as gender, age, activity level, and/or height, may be used to determine the most accurate set of bioimpedance sensors from which to collect metrics.

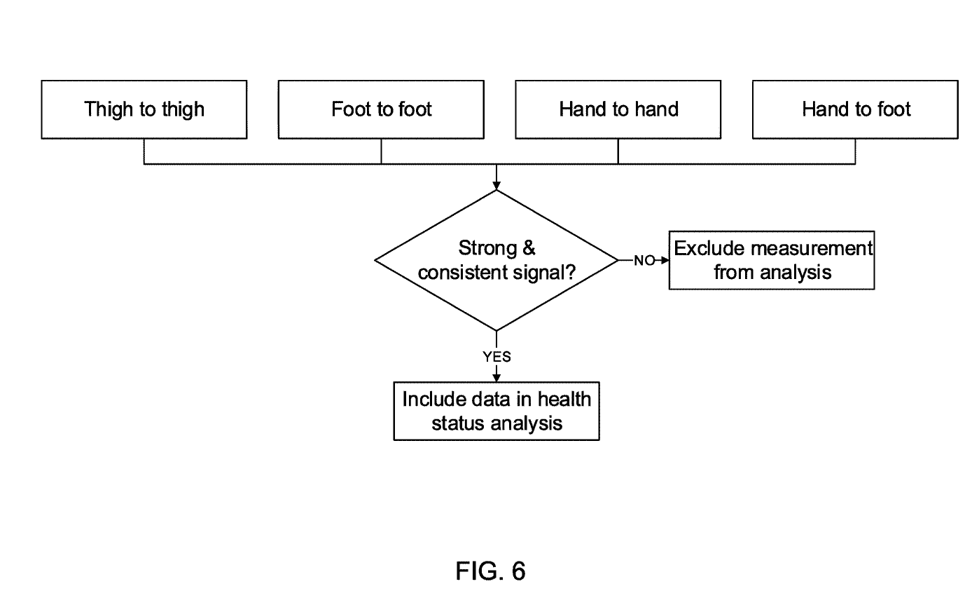

FIG. 5 illustrates bioimpedance sensors 410 and 412 on the seat which come in contact with a user's thighs when the user is seated on toilet 500. In addition, toilet 500 includes bioimpedance sensors 510 and 512 on scale 405 which a user may place in contact with the user's feet. Toilet 400 further includes handles 502 and 504 which comprise bioimpedance sensors 506 and 508. A user may place a right and a left hand on each of bioimpedance sensors 506 and 508 respectively to make measurements through the user's hands. Different pairs of bioimpedance sensors may be used either as a single pair or with other pairs of bioimpedance sensors. For example, a user may make a single measurement from hand to foot, foot to foot, hand to hand, or thigh to thigh. Alternatively, a user may make a hand to foot, a hand to hand, and a thigh to thigh measurement. The computer processor will use the metrics collected from all of these sources to make calculations and health status reports or only some if not all of the methods deliver at least an adequate signal. FIG. 6 is a flow chart showing an example decision tree which illustrates how multiple sources of bioimpedance data may be collected from different body parts as a user sits on the toilet as described herein. While the same physiological function is being measured by each set of sensors, only those that provide a signal that is strong enough and consistent enough to be considered reliable is included in health status analyses.

In situations when the user does not input gender data, urine flow rate can predict the gender of the user as females tend to have a greater urination rate than males. The reported data may inform the user that gender was predicted using this method and that data was interpreted accordingly. If the individual interpreting the data notes that the urination rate measurement assigned an inaccurate gender to the user, the computer processor may include a mechanism to recalculate the data using the correct gender. The user's gender may then be included in metric analyses.

In addition to variations that occur due to the specific body parts used to collect bioimpedance metrics, it has been shown that bioimpedance measurements are also less accurate if the user is moving. In some embodiments, including those shown in FIGS. 4 and 5, pressure sensors 410, and 412 on the seat and those of scale 405 may detect motion while the bioimpedance sensors are in use. If the user is moving too much to acquire an accurate bioimpedance measurement, the measurement may be rejected and a signal may indicate to the user that a repeat measurement is needed to collect useful data. The toilet may provide a signal, such as a verbal command or digital readout, instructing the user to remain motionless. The toilet may repeat the metric and then the signal until valid data is collected. As the skilled artisan will understand, this process may be performed for other metrics for which accuracy is compromised due to user movement.

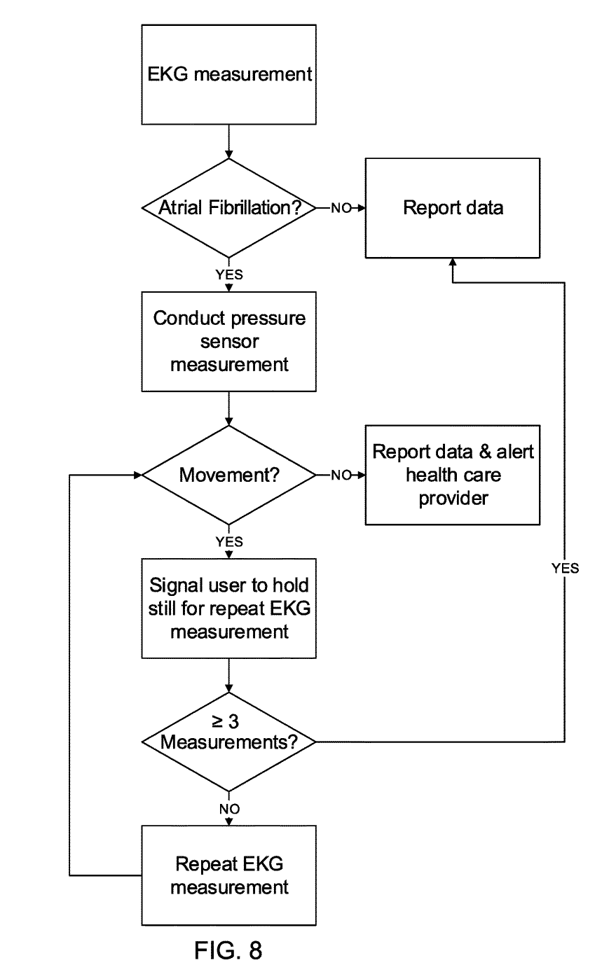

Collecting valid metrics may be especially important when the data suggests a serious pathology and either a false positive or a false negative could have serious consequences. For example, EKG measurements may suggest that the user is experiencing atrial fibrillation. The analysis system may provide a signal, such as a blinking light or a digital or verbal message, which tells the user to repeat the measurement. FIG. 8 is a flow chart illustrating an example in which an EKG measurement may indicate atrial fibrillation. The pressure sensor data assesses whether or not the user is moving such that the EKG data is not reliable. The user may be signaled to remain still while the EKG measurement is repeated. If atrial fibrillation is still detected when the measurement is conducted while the user sits still, the data is reported and identified as atrial fibrillation. Measurements that indicate severe health threats may be programmed to be repeated a minimum number of times before being reported to the user's health care provider even if reliable measurements are not acquired. In addition, the computer processor may be programmed to alert a health care provider immediately if it receives metrics that indicate a medical emergency.

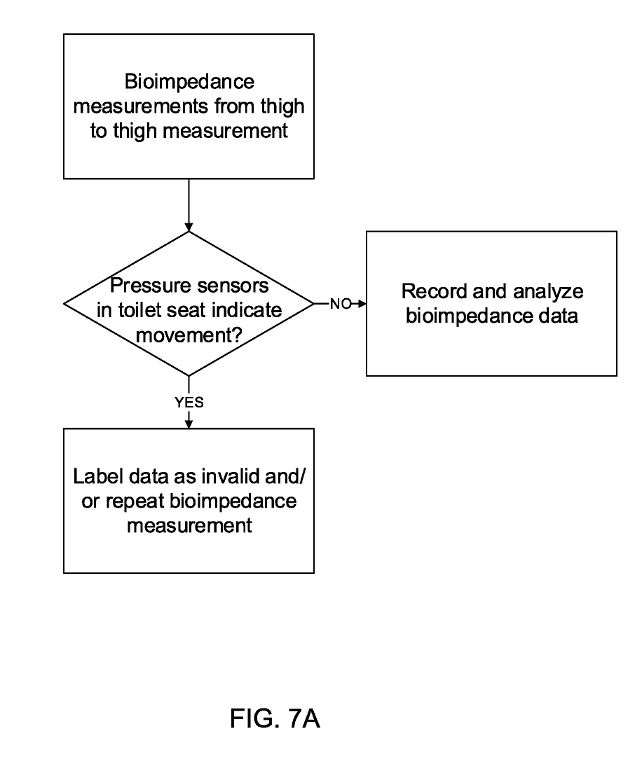

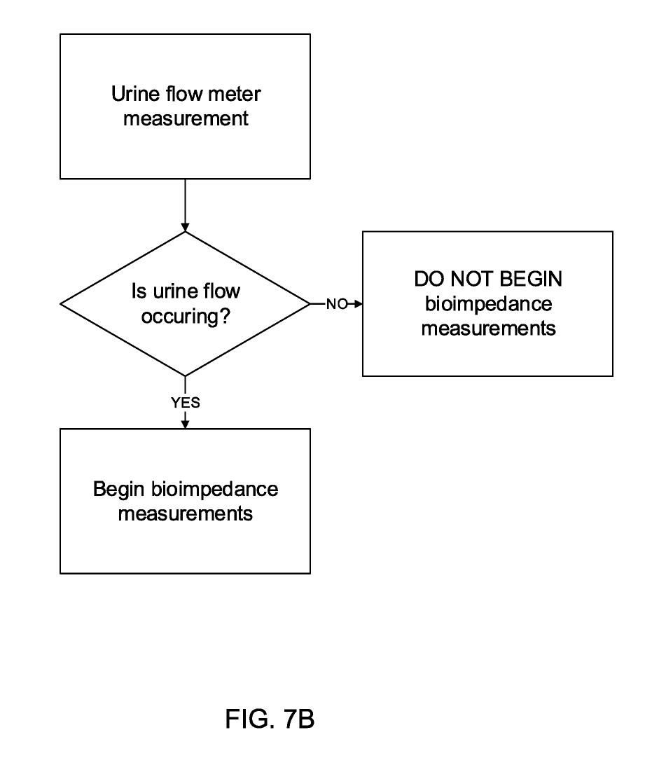

Alternatively, the motion detection system may be used to determine what type of metric to collect. For example, the motion detection may be used to determine whether to make a fast, single frequency bioimpedance measurement or to make a potentially more accurate multi-frequency measurement based on whether the user is relatively motionless or continues to move during measurements. This could be useful when the user is a small child or incoherent adult for which measurements may be difficult to obtain while the user is relatively still. The computer processor could be programmed to include bioimpedance data in calculations when a defined number of measurements fail due to excessive user motion. Alternatively, user input could indicate that data should not be rejected due to motion, rather, the computer processor could be programmed to use the best possible available measurement and simply flag the data to indicate that the user was moving during the measurement. FIG. 7A is a flow chart showing an example decision tree showing how bioimpedance data may be weighted based on movement data obtained through pressure sensors. Bioimpedance data collected while the user was moving as determined by pressure sensors is labeled as an invalid measurement. In some embodiments, the bioimpedance may be repeated in an attempt to acquire accurate measurements.

In addition, bioimpedance measurements are more accurate after urination is complete. In embodiments that include a flow meter or liquid level meter to identify when urination is complete, the computer processor could be programmed to reject bioimpedance measurements taken before urine flow ceases. Accordingly, a more accurate bioimpedance measurement may be used to calculate health parameters that inform assessment of user health status. FIG. 7B is a flow chart showing an example decision tree showing how bioimpedance data is valued based on whether or not the user is urinating during the measurement.

The heart rate may also be measured using a photoplethysmography (PPG) sensor which may be a finger clip 516 as shown in FIG. 5 or a reflectance mode PPG sensor available in certain smart phones. Alternatively, the PPG sensor may be a hand-held remote device or attachment that may be provided in an embodiment of the toilet disclosed herein. The heart rate may also be measured by electrocardiogram (EKG or ECG) measurements using a remote device held in each of the user's hands measuring conductance between the two hands. The electrocardiogram measurements may also be taken using wired connections from the user's right hand to one of the lower extremities, such as an electrode on the left thigh, left foot, right foot, plus an optional driven right leg connection. In addition, the heart rate may be measured using a pressure sensor against the skin or a seismometer. The heart rate may further be measured using stethoscope 514 as shown on toilet 500 which presses against the user's back when the user is seated and leaning back against the back of the toilet.

With these and other options for measuring heart rate, a method is needed for determining which measurement is the most relevant and accurate. Variables such as whether the user is wearing shoes, a thick jacket, holding the hand-held electrodes, or whether body fat in the thigh prevents an accurate PPG from the thigh may cause one method of measuring heart rate to be less effective than another. The computer processor may be programmed to ignore signals that are weak or inconsistent in favor of using data that is delivered by methods that provide better measurements. Furthermore, the computer processor may be programmed to process multiple metrics, including, but not limited to heart rate, over time and entered into an analysis system which builds a profile for the individual user. The analysis system may give priority to the most useful data and ignore the other when analyzing and combining data points and assembling a health status report. Over time, the analysis system may be programmed to ignore measurements from sources that have provided consistently poor data in the past.

In other examples, a user's fingers may be cold resulting in low blood perfusion in the fingers. Consequently, finger clip 516 may have difficulty detecting accurate PPG heart rate data. The analysis system may deprioritize this measurement in favor of another method of measuring the user's heart rate. Alternatively, if, on a particular day, bioimpedance measurements taken at low frequency (for example, approximately 1 kHz conduction) are indicative of a high resistance between two electrode contacts, the analysis system may ignore these measurements. In another example as described briefly above, a temperature sensor may be positioned near the stethoscope, for example, stethoscope 514 of FIG. 5. The temperature detected by the temperature sensor may provide an indication of whether stethoscope 514 is directly against the user's skin. If the measured temperature is significantly below normal body temperature, the user may either be wearing heavy clothing or not leaning against the stethoscope, either or which could prevent the stethoscope from obtaining an accurate reading. In such a situation, the analysis system may ignore the heart rate reading from the stethoscope. Instead, the analysis system may use heart rate data obtained from bioimpedance sensors 510 and 512 which may be positioned adjacent to the user's bare feet and providing a more accurate reading. As one of skill in the art will readily understand, heart rate is just a single embodiment of a type of health data that may be collected using the multiple sub-devices that may be present in various embodiments of the toilet and the data culled to identify the most useful and accurate measurements. This type of analysis may be accomplished with measurements of other physiological functions for which the toilet comprises multiple methods of detection.

In other embodiments, measurement of a physiological function may indicate that a health-related metric may not be estimated or calculated from this specific data set, even though the data set represents the metric that would normally be used to calculate the health-related metric. For example, measurements from stethoscope 514 or from pressure sensors located in scale 405 may suggest that the user is breathing abnormally fast. In this situation, heart rate measurements will not be recorded as “resting heart rate.” In contrast, heart rate measurements collected at a time when there is no indication of rapid breathing will be defined as a measurement of “resting heart rate.”

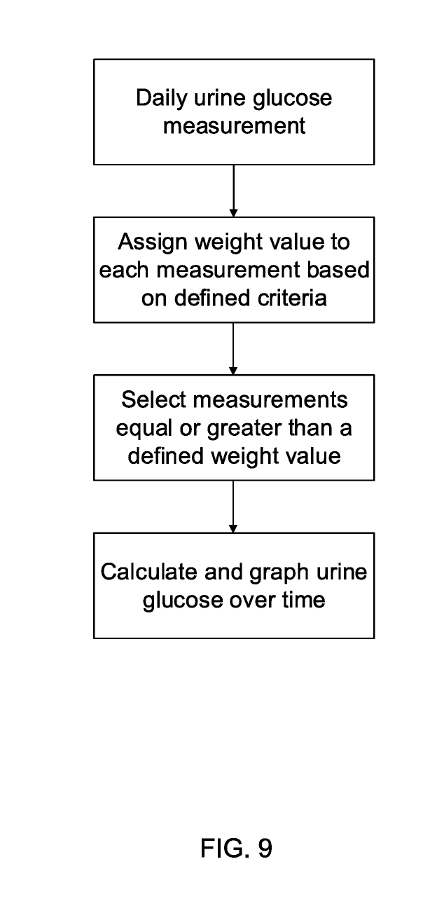

Multiple measurements of the same physiological function over time may be combined to produce a trending analysis. Unlike a clinical evaluation in which the measurements may always be valid, a trending analysis may include selected data points while others, deemed to be of insufficient quality, may be ignored. The value of trending analysis lies in the elimination of less accurate data and it provides a means for monitoring changes in a user's physiological functions over longer periods of time than can be obtained in a clinical setting. In other words, the clinical setting may provide one or several snapshots of an individual's health status, each of which are often given equal weight. A trending analysis provides more data points over a period of time and includes only data points that are deemed to be valid. FIG. 9 is a flow chart that illustrates an example in which daily urine glucose measurements are taken over time. Based on defined parameters, such as whether or not indications of dehydration are present, a weight value is assigned to each measurement. After a defined number of daily urine glucose measurements have been taken, only those measurements that have been assigned at least a minimum weight value to indicate a level of reliability are used to calculate and graph a trending analysis of urine glucose changes over time.

Furthermore, metrics from a medical device other than the toilet may be entered into the computer and used in calculations. For example, a user's health care provider may order clinical laboratory tests that are conducted by a hospital laboratory or tests performed by any medical devices other than the toilet describe herein. The data from sources other than the toilet may be entered into the computer and used to perform calculations. The calculations may be performed by combining metrics collected by the toilet with those from other sources. Alternatively, the calculations may perform separate calculations using either the metric collected by the toilet or the metric collected by the other source. For example, an analysis of a user's urine glucose may be performed by the toilet and in a hospital laboratory. By performing separate calculations, the metrics from the two sources may be compared. The computer processor may be programmed to produce a report of the calculations performed using metrics from either or both sources and provide an indication of the source of the metric(s) used to perform each calculation.

While specific embodiments have been illustrated and described above, it is to be understood that the disclosure provided is not limited to the precise configuration, steps, and components disclosed. Various modifications, changes, and variations apparent to those of skill in the art may be made in the arrangement, operation, and details of the methods and systems disclosed, with the aid of the present disclosure.

Without further elaboration, it is believed that one skilled in the art can use the preceding description to utilize the present disclosure to its fullest extent. The examples and embodiments disclosed herein are to be construed as merely illustrative and exemplary and not a limitation of the scope of the present disclosure in any way. It will be apparent to those having skill in the art that changes may be made to the details of the above-described embodiments without departing from the underlying principles of the disclosure herein.

We claim:

1. A health metering device, comprising:
a first medical device, wherein the first medical device comprises a toilet,
wherein the toilet performs a first metric and a second metric;
wherein the first metric is an indicator of a user's health status and the second metric in an indicator of the accuracy of the first metric;
wherein the toilet comprises a data communication port; and
a data collection system, the data collection system comprising a computer;
wherein the computer comprises a non-transitory computer readable medium that instructs the computer to:
receive and store data transmitted from the toilet through the data communication port; and
assign a weight value to the first metric, wherein the weight value is a function of the second metric and an indicator of the validity of the first metric.

2. The health metering device of claim 1, further comprising:
sensors for measuring bioimpedance between two body parts on a user,
sensors for measuring pressure,
sensors for measuring the user's blood pressure; and
sensors for measuring the user's heart rate.

3. The health metering device of claim 2, wherein the toilet further comprises at least one analytical device that measures at least one property of the user's biological waste.

4. The health metering device of claim 3, wherein the at least one property of the user's biological waste is selected from one or more of the following:
urine color, urine glucose concentration, urine urea concentration, urine creatinine concentration, urine specific gravity, urine protein concentration, urine electrolyte concentrations, urine pH, urine osmolality, urine human chorionic gonadotropin concentration, urine hemoglobin level, white blood cells in urine, red blood cells in urine, urine ketone body concentration, urine bilirubin concentration, urine urobilinogen concentration, urine free catecholamine concentration, urine free cortisol concentration, urine phenylalanine concentration, urine volume, fecal volume, fecal weight, fecal calprotectin level, fecal lactoferrin level, and fecal hemoglobin level.

5. The health metering device of claim 1, wherein the non-transitory computer readable medium instructs the computer to include the first metric in a calculation, wherein the result of the calculation is an indicator of the user's health status.

6. The health metering device of claim 1, wherein the non-transitory computer readable medium instructs the computer to exclude the first metric from calculations when the weight value is below a defined value.

7. The health metering device of claim 6, wherein the non-transitory computer readable medium instructs the computer to:
store multiple data sets that comprise of the first metric and the second metric, wherein the multiple data sets have been repeatedly collected from a user over a defined time period; and
perform a trending analysis on the first metric data points that were assigned a weight value that is greater than or equal to a defined value.

8. The health metering device of claim 6, wherein the toilet provides a signal instructing the user to modify the user's physical position, and then to repeat the first metric and the second metric until the first metric is assigned a weight value that indicates a minimum validity.

9. The health metering device of claim 1, wherein the first metric and the second metric are collected at different times.

10. The health metering device of claim 1, wherein the second metric is an indicator that the user's hydration is abnormal.

11. The health metering device of claim 10, wherein the first metric is an indicator of cardiovascular health.

12. The health metering device of claim 1, further comprising a data transmission device, wherein the data transmission device is inserted into the data communication port, and wherein the data transmission device transmits the first metric and second metric to the computer.

13. The health metering device of claim 1, wherein the non-transitory computer readable medium instructs the computer to send a signal to a person who is not the user, wherein the signal alerts the person that at least one of the first and the second metric is consistent with a possible medical emergency.

14. The health metering device of claim 1, wherein the values collected in the second metric determine which calculation is used to process the first metric.

15. The health metering device of claim 1, wherein the computer-readable medium instructs the computer to perform calculations using values from the first metric and the second metric, wherein the calculations determine the signal and the noise, and filter the noise from the signal collected from the first metric.

16. The health metering device of claim 1, wherein the computer-readable medium instructs the computer to prepare a differential diagnosis of the user's health status.

17. The health metering device of claim 1, wherein the toilet collects a plurality of metrics that employ a plurality of measuring mechanisms, wherein the plurality of metrics measure a same physiological parameter; and wherein the computer-readable medium instructs the computer to perform calculations using only the metric that was assigned the weight value that indicates a highest validity.

18. The health metering device of claim 1,
wherein the computer-readable medium instructs the computer to record a metric that was collected using a second medical device and to perform calculations using the both metric collected by the second medical device, and a first metric and a second metric collected by the toilet.

19. The health metering device of claim 18, wherein the metric collected from the second medical device measures the same parameter as the first metric measured by the toilet.

20. The health metering device of claim 18,
Wherein the computer-readable medium instructs the computer to prepare a report, wherein the report identifies whether metrics from the toilet, metrics from the second medical device, or metrics from both the toilet and the second medical device were used to perform a calculation.
